(19) United States
(12) Patent Application Publication
(10) Pub. No.: US 2017/0202185 A1
(43) Pub. Date: Jul. 20, 2017

(54) UNMANNED LIVESTOCK MONITORING SYSTEM AND METHODS OF USE

(71) Applicant: Dinklage Feed Yards, Inc., Sidney, NE (US)

(72) Inventors: Timothy Rex Trumbull, Torrington, WY (US); Susan Rene Myrtle, Torrington, WY (US)

(21) Appl. No.: 15/000,018

(22) Filed: Jan. 18, 2016

(51) Int. Cl. A01K 29/00; B64C 39/02; A61B 5/0205; A61B 5/01; A61B 5/145

(52) U.S. Cl. CPC A01K 29/005; A61B 5/01; A61B 5/14539; A61B 5/14542; A61B 5/02055; A61B 5/14507; B64C 39/024; A61B 2503/40; A61B 5/08

(57) ABSTRACT

The present invention relates in general to the field of animal husbandry, and more specifically, to a livestock monitoring system utilizing an unmanned aerial vehicle (“UAV”) and methods of using such systems. The system and method of the present invention for monitoring the health and welfare of livestock comprises six primary components: (1) at least one UAV; (2) a plurality of cameras and sensors; (3) a transmitter; (4) a receiver; (5) a server connected to a computer system for receiving images, video, and data from the plurality of cameras and sensors; and (6) a display for viewing in real-time images, video and data obtained from the plurality of cameras and sensors for monitoring the condition of livestock on a farm or ranch. The purpose of the invention is to provide a convenient and cost-efficient system and method for monitoring the condition of livestock to obtain information in real-time about the behavioral and physiological states of individual animals.

### FIELD OF THE INVENTION

The present invention relates in general to the field of animal husbandry, and more specifically, to a livestock monitoring system utilizing an unmanned aerial vehicle (“UAV”) and methods of using such systems. The purpose of the invention is to provide a convenient and cost-efficient system and method for monitoring the condition of livestock to obtain information in real-time about the behavioral and physiological states of individual animals. In particular, this information may be used to determine the health and welfare of livestock. A further purpose of the invention is to provide an unmanned livestock monitoring system and method that determines feed and water quality for the livestock. An additional purpose of the invention is to provide an unmanned livestock monitoring system and method that locates stray animals and controls the movement of livestock when sorting between pens or arranging for transport and shipping.

### BACKGROUND OF THE INVENTION

Historically in the United States, the cattle industry may be best illustrated by the large cattle drives of the 1880s, where cattle were herded from the south-central United States to rail centers such as Abilene, Kans. and Cheyenne, Wyo. During the decades after the United States Civil War, over 40,000 men, known as cowboys, were seasonally hired to round-up and drive cattle on the slow and dangerous journey to the train stations. Between the years of 1866 to 1888, over 4,000,000 head of cattle were driven over the vast open ranges of the prairie, typically in herds between 1,000 to 10,000 animals Cowboys not only were needed to guide the cattle to their proper destination, but also to locate strays, check for disease, find good grazing land and water, and to offer protection from wild animals and/or rustlers. Once reaching such rail centers, cattle were transported live to urban areas such as Chicago, where they were slaughtered, processed, and shipped to consumers out East.

The end of the open range due to legislation, homesteaders, and especially barbed wire spelled the end of the long cattle drive in the late 1880s. Nevertheless, ranching techniques were adopted to create controlled, fenced ranges where the livestock could be fed, watered, and protected by permanently employed cowboys. Notably, in 1900 the average farm/ranch size in the United States was 147 acres. Over time, cattle raising became a regular business, with Easterners and even Europeans investing in cattle. The cattle industry began to grow exponentially, wherein the number of total U.S. calves doubled by 1900 and then doubled again by 1970. Today, most farms/ranches are at least 1,100 acres, and many are five and ten times that size. Current numbers show that the U.S. produced 89.8 million head of cattle in 2014, generating over $44 billion in farm gate receipts.

As the size of farms/ranches and livestock herds has increased drastically over the past 100 years, the ability of farmers/ranchers to personally monitor the condition of their livestock herds has also grown in difficulty and expense. Human visual observation to monitor the health, fertility and condition of individual animals has become impractical and cost-prohibitive due to the large number of animals and vast distances encompassing a farm/ranch. In response to these evolving conditions, some farmers/ranchers have turned towards performing such monitoring and/or managing through the use of electronic tags associated with individual animals. Electronic documentation and verification involves the use of machine readable/writeable tags, in the manner of conventionally-known ear tags, to be implanted or internally carried by the animal. Such tags may be tied to a database identifying and recording various events during the livestock production and processing cycle, for instance, the receipt of livestock at a feedlot from another facility, medicines or other treatments applied, feeding protocols, shipping and meat processing. Particularly, the use of machine-readable radio frequency identification (“RFID”) tags enables some automation of recognizing the presence of a specific animal within the range of an RFID interrogator. However, RFID tags have a limited range, requiring an animal to be contained within a squeeze chute or other restraint for identification and assurance of a reliable tag reading. Unfortunately, in the real-world such methods are impractical, time-consuming, and require additional personnel.

Presently there is no system that can do any of the real-time condition based monitoring of livestock necessary to protect, promote, and improve the welfare of the animals without requiring a farmer or rancher to physically be present. With the increasing scale of farming, it has become more difficult—if not impossible—for stockmen to rely upon traditional observation methods to accurately monitor livestock herds. Thus, a desire remains to develop a convenient, time-saving and cost-efficient system and method for monitoring the condition of livestock to obtain information in real-time about the location, behavioral and physiological states of individual animals.

### BRIEF SUMMARY OF THE INVENTION

Therefore, it is a principal object, feature, and/or advantage of the present invention to overcome the aforementioned deficiencies in the art and provide a convenient and cost-efficient system and method for monitoring the condition of livestock.

An additional object, feature, and/or advantage of the present invention is to provide an unmanned system and method for monitoring the condition of livestock that utilizes a UAV.

Another object, feature, and/or advantage of the present invention is to provide an unmanned system and method for monitoring the condition of livestock that obtains information in real-time.

Yet another object, feature, and/or advantage of the present invention is to provide an unmanned system and method for monitoring the condition of livestock that obtains information about the behavioral and physiological states of individual animals.

A further object, feature, and/or advantage of the present invention is to provide an unmanned system and method for monitoring the condition of livestock that obtains information about the health, welfare and fertility states of individual animals.

A still further object, feature, and/or advantage of the present invention is to provide an unmanned system and method for monitoring the condition of livestock that obtains information about the rate of gain, feeding patterns and water intake levels of individual animals.

Another object, feature, and/or advantage of the present invention is to provide an unmanned system and method for monitoring the condition of livestock that identifies illnesses, the severity of any illness and animals with low or high body temperature readings.

Yet another object, feature, and/or advantage of the present invention is to provide an unmanned system and method for monitoring the condition of livestock that identifies excessive animal behaviors.

A further object, feature, and/or advantage of the present invention is to provide an unmanned system and method for monitoring the condition of livestock that obtains information in real-time about feed conditions, feed quality, feed distribution, feed consumption, feed and water availability and water quality for the animals.

A still further object, feature, and/or advantage of the present invention is to provide an unmanned system and method for monitoring the condition of livestock that locates animals in distress and/or strays.

Another object, feature, and/or advantage of the present invention is to provide an unmanned system and method for monitoring the condition of livestock that controls the movement of animals when sorting between pens or arranging for transport and shipping.

These and/or other objects, features, and/or advantages of the present invention will be apparent to those skilled in the art. The present invention is not to be limited to or by these objects, features, and advantages. No single aspect need provide each and every object, feature, or advantage.

According to one aspect of the present invention, a system and method for monitoring the condition of livestock, particularly, for monitoring the health and welfare of the livestock, is provided. The system and method of the present invention for monitoring the health and welfare of livestock comprises six primary components: (1) at least one UAV; (2) a health and welfare assessment device(s); (3) a transmitter; (4) a receiver; (5) a server connected to a computer system; and (6) a display for viewing in real-time health and welfare data obtained from the health and welfare assessment device(s) for monitoring the condition of livestock on a farm or ranch. Particularly, the health and welfare assessment device(s) may be onboard the UAV and comprise one or more camera(s) and a plurality of sensors for monitoring the health and welfare of livestock. The health and welfare assessment device(s) may obtain real-time health and welfare data on the condition of livestock such as assessing an animal's temperature before/after it shows signs of illness, the onset of disease and the identity/contagiousness of any disease. After viewing on the display the health and welfare data obtained by the health and welfare assessment device(s), a farm or ranch manager may take corrective action to safeguard the health and welfare of his/her livestock.

According to another aspect of the present invention, a system and method for monitoring the condition of livestock, particularly, for monitoring feed and water conditions in a feed lot, confinement building and/or pasture is provided. The system and method of the present invention for monitoring feed and water conditions comprises six primary components: (1) at least one UAV; (2) a feed and water assessment device(s); (3) a transmitter; (4) a receiver; (5) a server connected to a computer system; and (6) a display for viewing in real-time feed and water data obtained from the feed and water assessment device(s) for monitoring feed and water conditions in a feed lot, confinement building and/or pasture. Particularly, the feed and water assessment device(s) may be onboard the UAV and comprise one or more camera(s) and a plurality of sensors for monitoring the feed and water conditions in a feed lot, confinement building or pasture. The feed and water assessment device(s) may obtain real-time feed and water data such as determining feed and water availability, cleanliness, quality and freshness. After viewing on the display the feed and water data obtained by the feed and water assessment device(s), a farm or ranch manager may take corrective action to promote the growth and vitality of livestock on a farm or ranch.

According to a further aspect of the present invention a system and method for monitoring the condition of livestock, particularly, for determining the location and controlling the movement of livestock is provided. The system and method of the present invention for determining the location and controlling the movement of livestock comprises six primary components: (1) at least one UAV; (2) an animal locator and herding device(s); (3) a transmitter; (4) a receiver; (5) a server connected to a computer system; and (6) a display for viewing in real-time animal location data obtained from the animal locator and herding device(s) for determining the location and controlling the movement of livestock on a farm or ranch. Particularly, the animal locator and herding device device(s) may be onboard the UAV, wherein the animal locator and herding device(s) may comprise one or more camera(s) and a plurality of sensors for determining the location and controlling the movement of livestock. The animal locator and herding device(s) may obtain in real-time animal location data for any particular animal of a livestock herd in a feed lot, confinement building or pasture. Thus, after viewing on the display the animal location data obtained by the animal locator and herding device(s), a farm or ranch manager may be able to locate animals in distress and create controlled movement of the livestock herd and/or individual animals between pens and for loading and transportation purposes.

Different aspects may meet different objects of the invention. Other objectives and advantages of this invention will be more apparent in the following detailed description taken in conjunction with the figures. The present invention is not to be limited by or to these objects or aspects.

### DESCRIPTION OF FIGURES

FIGS. 1-6 represent examples of systems of the present invention for monitoring the condition of livestock utilizing a UAV, and a method of monitoring livestock.

FIG. 1 is an elevational view and schematic representation of a farm/ranch office and pasture with which the system and method of the present invention for monitoring the health and welfare of the livestock would be utilized.

FIG. 2 is a flow chart of a system and method of the present invention for monitoring the health and welfare of livestock.

FIG. 3 is an elevational view and schematic representation of a farm/ranch office and a feed lot with which the system and method of the present invention for monitoring feed and water conditions for livestock would be utilized.

FIG. 4 is a flow chart of a system and method of the present invention for monitoring feed and water conditions for livestock.

FIG. 5 is an elevational view and schematic representation of a farm/ranch office and a pasture and corral with which the system and method of the present invention for determining the location and controlling the movement of livestock would be utilized.

FIG. 6 is a flow chart of a system and method of the present invention for determining the location and controlling the movement of livestock.

### DETAILED DESCRIPTION OF THE INVENTION

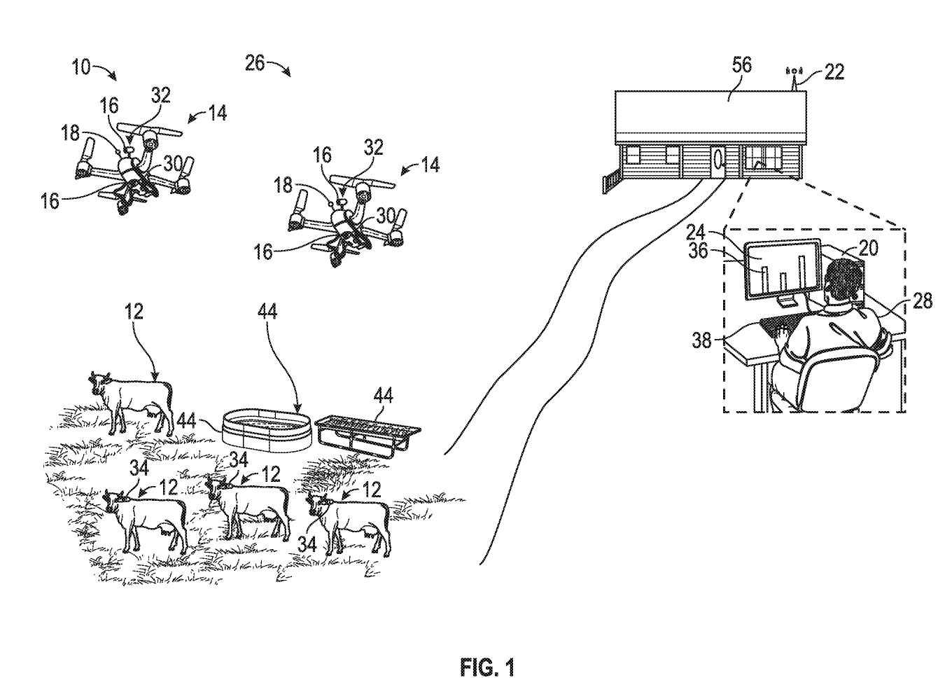

FIG. 1 illustrates one aspect of the system and method of the present invention for monitoring the condition of livestock (10), particularly, for monitoring the health and welfare of the livestock. Used herein, the term “livestock” (12) refers to any animal or group of animals which is intended to be monitored and/or managed, regardless of whether the animal(s) are domesticated, semi-domesticated or wild, and regardless of the environment in which the animal may be found, for example, in a commercial farming/ranching operation or in a wild environment.

As shown in FIG. 1, the system and method of the system and method of the present invention (10) for monitoring the health and welfare of livestock (12) comprises six primary components, including but not limited to: (1) at least one UAV (14) and/or unmanned aircraft system (“UAS”) which includes ground stations and other elements in addition to the UAV; (2) a health and welfare assessment device(s) (16) onboard the UAV and/or located remotely from the UAV; (3) a transmitter (18) onboard the UAV; (4) a receiver (22) for receiving health and welfare data from the transmitter; (5) a server (20) for receiving the health and welfare data from the receiver and further connected to a computer system; (6) a display (24) for viewing in real-time the health and welfare data obtained from the health and welfare assessment device(s) for monitoring the condition of livestock on a farm or ranch.

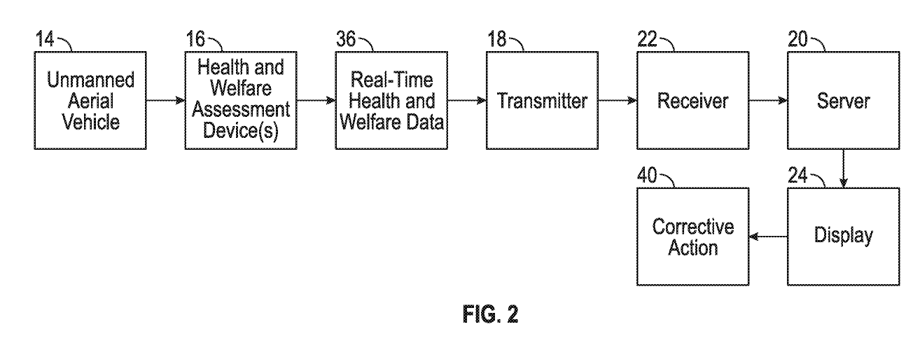

Illustrated in FIGS. 1-2, the first primary component of the system and method of the present invention (10) for monitoring the health and welfare of livestock (12) comprises at least one UAV (14) or UAS. The UAV (14) may be of a type standardly used in the industry. Depending upon the intended use of the livestock monitoring system (10) (i.e., whether for use on a large farm/ranch or a confined feedlot), a specific type of UAV (14) may be chosen by an operator (28) (e.g., farm or ranch manager). For instance, if the intended use is for a smaller area the operator may choose a rotary UAV that typically has between two to ten rotors. Rotary UAVs have limited battery efficiency and are therefore best utilized for relatively smaller areas (e.g., less than 100 acres). These rotors provide optimal stability, control and maneuverability for individual animal assessment on a feedlot, confinement building, pasture, or smaller area. Alternatively, if the intended use is for a large area covering many acres the operator may choose a fixed-wing and/or a blended fuselage-wing UAV such as an all-lifting body. A fixed-wing UAV operates like a small model airplane and may be fabricated using lightweight foam. Because of its minimal weight, a fixed-wing UAV is more efficient in battery usage and is therefore best utilized for larger areas (e.g., over 100 acres) and may travel at speeds in excess of 100 mph. Both rotary and fixed-wing UAVs, used alone or in combination, may be incorporated into the system and method of the present invention for monitoring the health and welfare of livestock. It is to be understood that the precise type and style of UAV is not a limitation to the present invention. The foregoing UAVs are described for illustrative purposes only as it is contemplated other UAVs commonly used in the industry may also be used by the system and method of the present invention.

As shown in FIGS. 1-2, the second primary component of the system and method of the present invention (10) for monitoring the health and welfare of livestock (12) comprises the health and welfare assessment device(s) (16). The health and welfare assessment device(s) (16) may be onboard the UAV (14), wherein the health and welfare assessment device(s) (16) may comprise one or more camera(s) (30) for capturing still images and video. The health and welfare assessment device(s) (16) may further comprise a plurality of sensors (32) onboard the UAV (14) for monitoring the health and welfare of livestock (12). The health and welfare assessment device(s) (16) may also include remote sensors (34), wherein remote sensors (34) may be located in ear tags, head collars, leg attachments, confinement buildings, corrals, feeding outlets, watering outlets, pastures, and/or combinations thereof. The remote sensors (34) may comprise unique identifiers associated with a particular location and/or purpose for the remote sensor (34). The remote sensors (34) may also be connected via a bus architecture so that additional sensors may be added or removed as required. The remote sensors (34) may be reusable so that they can be reprogrammed and used at another location or for another purpose. It is contemplated that an array of cameras (30) and sensors (32, 34) in a variety of locations may be utilized as health and welfare assessment device(s) (16) by the present invention, including but not limited to, electro-optical/infrared imaging, thermal imaging, high definition video and still imaging, multiple object tracking, geo-location, atmospheric soundings, soil moisture determination, biological phenomena observation, barometric pressure recordings, temperature recordings, humidity recordings, meteorological recordings, chemical determination, laser spectroscopy, hyperspectral imaging, RFID tags (e.g., ear tags, implants), high frequency tags (e.g., ear tags, implants), gas analyzers, spatio-temporal image change detection, precision agriculture, pest detection, GPS, target tracking, pH determination, pollution monitoring, and/or plant identification.

The health and welfare assessment device(s) (16) may obtain real-time health and welfare data (36) on the condition of livestock (12) daily, hourly and/or multiple times per day/night. Health and welfare data (36) may include, but is not limited to, still images and video captured by the one or more camera(s) (30) and information obtained from the plurality of sensors (32) and remote sensors (34). For instance, health and welfare data (36) may include assessing an animal's temperature before/after it shows signs of illness, the onset of disease and the identity/contagiousness of any disease. Health and welfare data (36) may further include treatment results and quarantine monitoring of sick livestock. Health and welfare data (36) may also include bedding availability and cleanliness, mineral offerings and drug requirements. Health and welfare data (36) may further include detecting fertility status in breeding animals, the pH of biological fluids, blood flow or blood oxygenation, vocalization and respiration recognition, breath and saliva contents, weather conditions, environmental temperatures and biosecurity surveillance. Biosecurity surveillance is the process of systematically collecting, analyzing and interpreting information about the presence or absence of pests, diseases and unwanted organisms. Health and welfare data (36) may also include observations for calculating rate of gain, identifying eating patterns and viewing water intake levels for individual animals. Health and welfare data (36) may further identify eating disorders in livestock (e.g., animals not eating or drinking, animals overeating or overdrinking), poisonous plants within the vicinity of the livestock herd, excessive animal behaviors, downers and combinations of the foregoing.

As further shown in FIGS. 1-2, the third primary component of the system and method of the present invention (10) for monitoring the health and welfare of livestock (12) comprises the transmitter (18) (or transceiver). The transmitter (18) may be onboard the UAV (14) and wirelessly communicate the health and welfare data (36) obtained from the health and welfare assessment device(s) (16). Wireless transmitters utilized in the present invention may be any commercially available type, wherein the precise wireless transmitter not being a limitation of the present invention. The transmitter (18) may include a built-in antennae for transmission of the health and welfare data (36) obtained from the health and welfare assessment device(s) (16). The UAV (14) may further comprise a processor and a guidance system (not shown). The processor may comprise means for performing object detection and/or tracking, and further comprise means for on-board processing of the health and welfare data (36) prior to transmission.

As further shown in FIGS. 1-2, the fourth primary component of the system and method of the present invention (10) for monitoring the health and welfare of livestock (12) comprises the receiver (22) (or transceiver). The receiver (22) may wirelessly receive the health and welfare data (36) communicated from the transmitter (18) onboard the UAV (14) via a local wireless link and/or using a satellite link. The remote sensors (34) may also be wirelessly linked to the receiver (22). If the receiver (22) is a transceiver, the transceiver may wirelessly send commands from the operator (28) via the computer system (38) for operating the guidance system of the UAV (14) and health and welfare assessment device(s) (16), wherein the processor onboard the UAV (14) may execute the received commands.

As further shown in FIGS. 1-2, the fifth primary component of the system and method of the present invention (10) for monitoring the health and welfare of livestock (12) comprises the server (20). The server (20) may be connected wirelessly or via cables to the receiver (22). The receiver (22) may communicate the health and welfare data (36) received from the transmitter (18) to the server (20). The server (20) may be connected to a computer system (38), wherein the operator (28) may transmit commands via the computer system (38) to the guidance system of the UAV (14) for maneuvering the UAV (e.g., adjusting altitude, speed, heading, and positioning) and controlling the health and welfare assessment device(s). UAVs (14) of the present invention may be controlled by the operator (28) at all times or have built-in control and/or guidance systems to perform low level human pilot duties such as speed and flight path stabilization, and simple automated navigation functions such as waypoint following.

As further shown in FIGS. 1-2, the sixth primary component of the system and method of the present invention (10) for monitoring the health and welfare of livestock (12) comprises the display (24) for viewing in real-time the health and welfare data (36) obtained by the health and welfare assessment device(s) (16). The display (24) may be connected to the computer system (38), wherein the computer system (38) may be configured to automatically analyze and selectively create a concise summary and visualization on the display (24) that highlights notable events concerning the health and welfare of the livestock (12). The computer system (38) may further comprise a memory (not shown) for storing health and welfare data (36) obtained from the health and welfare assessment device(s) (16). Examples of computer systems (38) that may be utilized by the livestock monitoring system and method of the present invention (10) include, but are not limited to, a mainframe, a personal computer (PC), a cable set-top box, a television microprocessor, a handheld computer, a lap-top computer, a tablet, a smart-phone device, and/or combinations thereof. The server (20) and computer system (38) may be connected to a satellite or a network such as the Internet or a local area network.

After viewing on the display (24) the health and welfare data (36) obtained by the health and welfare assessment device(s) (16), the operator (28) may take corrective action to safeguard the health and welfare of livestock (12) on a farm or ranch.

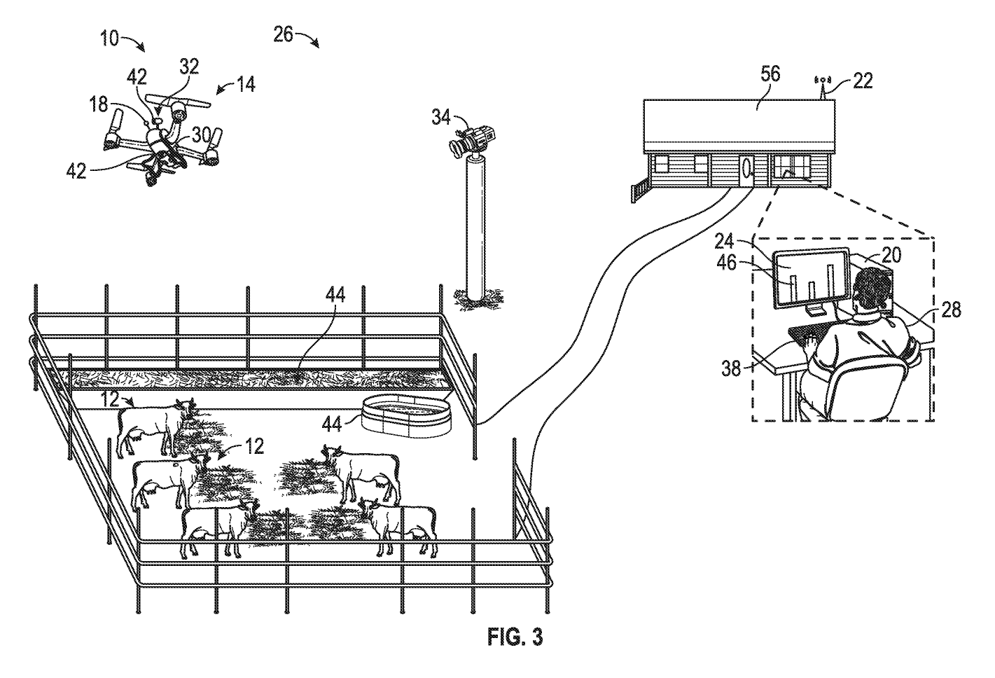

FIG. 3 illustrates another aspect of the system and method of the present invention (1) for monitoring the condition of livestock (12), particularly, for monitoring feed and water conditions (44) in a feed lot, confinement building and/or pasture. As shown in FIG. 3, the present invention (10) for monitoring feed and water conditions (44) comprises six primary components, including but not limited to: (1) at least one UAV (14) and/or UAS; (2) a feed and water assessment device(s) (42) onboard the UAV and/or located remotely from the UAV; (3) a transmitter (18) onboard the UAV; (4) a receiver (22) for receiving feed and water data from the transmitter; (5) a server (20) for receiving the feed and water data from the receiver and further connected to a computer system; and (6) a display (24) for viewing in real-time feed and water data obtained from the feed and water assessment device(s) for monitoring feed and water conditions in a feed lot, confinement building and/or pasture.

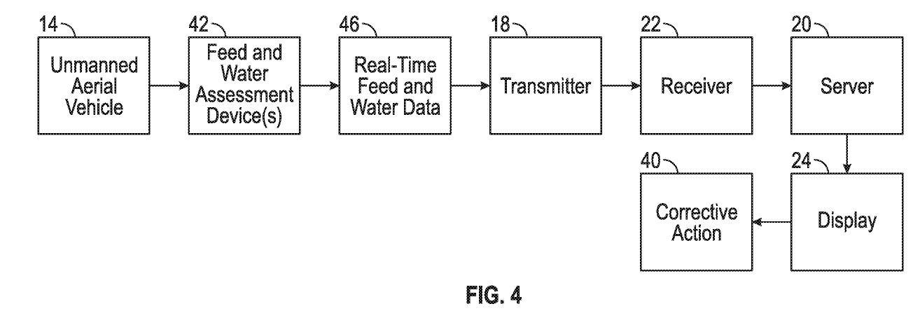

Illustrated in FIGS. 3-4, the first primary component of the system and method present invention (10) for monitoring feed and water conditions (44) in a feed lot, confinement building and/or pasture comprises at least one UAV (14) or UAS. The UAV (14) may be of a type standardly used in the industry. Depending upon the intended use of the livestock monitoring system (10) (i.e., whether for use on a large farm/ranch or a confined feedlot), a specific type of UAV (14) may be chosen by an operator (28) (e.g., farm or ranch manager). As mentioned previously, if the intended use is for a smaller area the operator may choose a rotary UAV that typically has between two to ten rotors. Rotary UAVs have limited battery efficiency and are therefore best utilized for relatively smaller areas (e.g., less than 100 acres). These rotors provide optimal stability, control and maneuverability for individual animal assessment on a feedlot, confinement building, pasture, or smaller area. Alternatively, if the intended use is for a large area covering many acres the operator may choose a fixed-wing and/or a blended fuselage-wing UAV such as an all-lifting body. A fixed-wing UAV operates like a small model airplane and may be fabricated using lightweight foam. Because of its minimal weight, a fixed-wing UAV is more efficient in battery usage and is therefore best utilized for larger areas (e.g., over 100 acres) and may travel at speeds in excess of 100 mph. Both rotary and fixed-wing UAVs, used alone or in combination, may be incorporated into the system and method of the present invention for monitoring the health and welfare of livestock. It is to be understood that the precise type and style of UAV is not a limitation to the present invention. The foregoing UAVs are described for illustrative purposes only as it is contemplated other UAVs commonly used in the industry may also be used by the system and method of the present invention.

As shown in FIGS. 3-4, the second primary component of the system and method of the present invention for monitoring feed and water conditions (44) in a feed lot, confinement building and/or pasture comprises the feed and water assessment device(s) (42). The feed and water assessment device(s) (42) may be onboard the UAV (14), wherein the feed and water assessment device(s) (44) may comprise one or more camera(s) (30) for capturing still images and video. The feed and water assessment device(s) (42) may further comprise a plurality of sensors (32) onboard the UAV (14) for monitoring the feed and water conditions (44) in a feed lot, confinement building and/or pasture. The feed and water assessment device(s) (42) may also include remote sensors (34), wherein remote sensors (34) may be located in confinement buildings, corrals, feeding outlets, watering outlets, pastures, and/or combinations thereof. The remote sensors (34) may comprise unique identifiers associated with a particular location and/or purpose for the remote sensor (34). The remote sensors (34) may also be connected via a bus architecture so that additional sensors may be added or removed as required. The remote sensors (34) may be reusable so that they can be reprogrammed and used at another location or for another purpose. It is contemplated that an array of cameras (30) and sensors (32, 34) in a variety of locations may be utilized as feed and water assessment device(s) (42) by the present invention (10), including but not limited to, electro-optical/infrared imaging, thermal imaging, high definition video and still imaging, multiple object tracking, geo-location, atmospheric soundings, soil moisture determination, biological phenomena observation, barometric pressure recordings, temperature recordings, humidity recordings, meteorological recordings, chemical determination, laser spectroscopy, hyperspectral imaging, RFID tags (e.g., ear tags, implants), high frequency tags (e.g., ear tags, implants), gas analyzers, spatio-temporal image change detection, precision agriculture, pest detection, GPS, target tracking, pH determination, pollution monitoring, plant identification, and combinations of the foregoing.

The feed and water assessment device(s) (42) may obtain real-time feed and water data (46) in a feed lot, confinement building and/or pasture daily, hourly and/or multiple times per day/night. Feed and water data (46) may include, but is not limited to, still images and video captured by the one or more camera(s) (30) and information obtained from the plurality of sensors (32) and remote sensors (34). For instance, feed and water data (46) may also include monitoring the proper distribution of feed and feed delivery patterns. Feed and water data (46) may further include identifying the amount of feed available, at any given time, at any given location, and at any specific time of day/night. Feed and water data (46) may also include observing animal response in relationship to feed delivery (e.g., aggressiveness or disinterest). Feed and water data (46) may further include determining feed availability, cleanliness, quality and freshness. Feed and water data (46) may further include determining water availability, cleanliness, quality, freshness and combinations of the foregoing.

As further shown in FIGS. 3-4, the third primary component of the system and method of the present invention (10) for monitoring feed and water conditions (44) in a feed lot, confinement building and/or pasture comprises the transmitter (18) (or transceiver). The transmitter (18) may be onboard the UAV (14) and wirelessly communicate the feed and water data (46) obtained from the feed and water assessment device(s) (42). As mentioned previously, wireless transmitters utilized in the present invention may be any commercially available type, wherein the precise wireless transmitter not being a limitation of the present invention. The transmitter (18) may include a built-in antennae for transmission of the feed and water data (46) obtained from the feed and water assessment device(s) (42). The UAV (14) may further comprise a processor and a guidance system (not shown). The processor may comprise means for performing object detection and/or tracking, and further comprise means for on-board processing of the feed and water data (46) prior to transmission.

As further shown in FIGS. 3-4, the fourth primary component of the system and method of the present invention (10) for monitoring feed and water conditions (44) in a feed lot, confinement building or pasture comprises a receiver (22) (or transceiver). The receiver (22) may wirelessly receive the feed and water data (46) communicated from the transmitter (18) onboard the UAV (14) via a local wireless link and/or using a satellite link. The remote sensors (34) may also be wirelessly linked to the receiver (22). If the receiver (22) is a transceiver, the transceiver may wirelessly send commands from the operator (28) via the computer system (38) for operating the guidance system of the UAV (14) and feed and water assessment device(s) (42), wherein the processor onboard the UAV (14) may execute the received commands.

As further shown in FIGS. 3-4, the fifth primary component of the system and method of the present invention (10) for monitoring the feed and water conditions (44) of livestock (12) comprises the server (20). The server (20) may be connected wirelessly or via cables to the receiver (22). The receiver (22) may communicate the feed and water data (46) received from the transmitter (18) to the server (20). The server (20) may be connected to the computer system (38), wherein the operator (28) may transmit commands via the computer system (38) to the guidance system of the UAV (14) for maneuvering the UAV (e.g., adjusting altitude, speed, heading, and positioning) and controlling the feed and water assessment device(s) (42). UAVs (14) of the present invention may be controlled by the operator (28) at all times or have built-in control and/or guidance systems to perform low level human pilot duties such as speed and flight path stabilization, and simple automated navigation functions such as waypoint following.

As further shown in FIGS. 3-4, the sixth primary component of the system and method of the present invention (10) for monitoring feed and water conditions (44) in a feed lot, confinement building and/or pasture comprises a display (24) for viewing in real-time the feed and water data (46) obtained by the feed and water assessment device(s) (42). The display (24) may be connected to the computer system (38), wherein the computer system (38) may be configured to automatically analyze and selectively create a concise summary and visualization on the display (24) that highlights notable events concerning the livestock's (12) feed and water conditions (44) in a feed lot, confinement building and/or pasture. The computer system (38) may further comprise a memory (not shown) for storing feed and water data (46) obtained from the feed and water assessment device(s) (42). Examples of computer systems (38) that may be utilized by the livestock monitoring system and method of the present invention (10) include, but are not limited to, a mainframe, a personal computer (PC), a cable set-top box, a television microprocessor, a handheld computer, a lap-top computer, a tablet, a smart-phone device, and/or combinations thereof. The server (20) and computer system (38) may be connected to a satellite or a network such as the Internet or a local area network.

After viewing on the display (24) the feed and water data (46) obtained by the feed and water assessment device(s) (42), the operator (28) may take corrective action to promote the growth and vitality of livestock (12) on a farm or ranch.

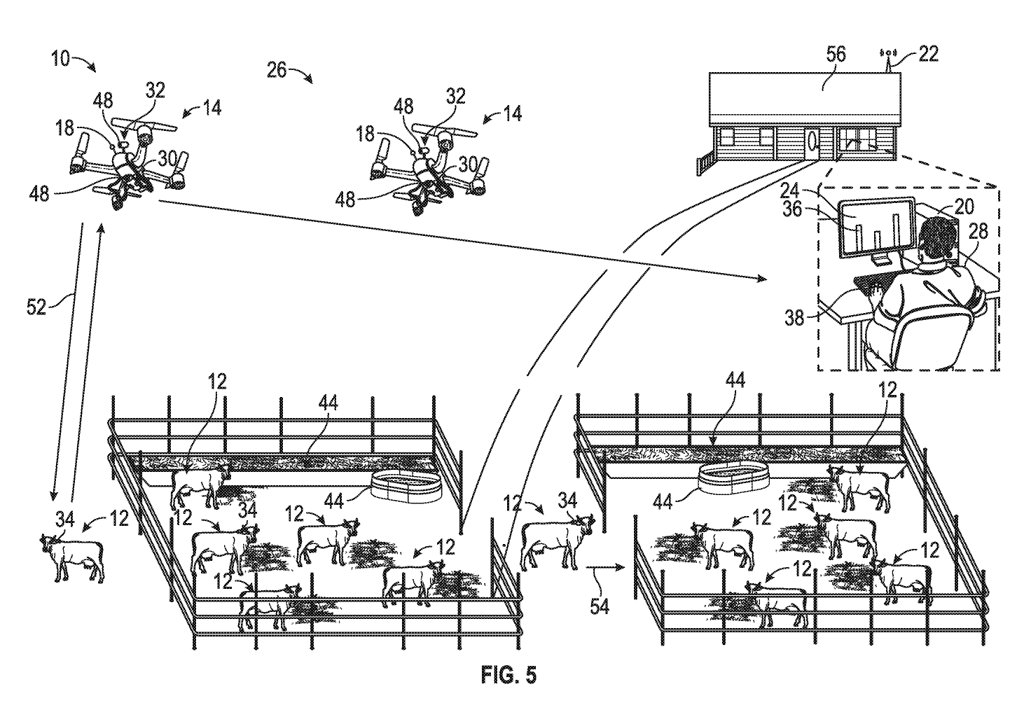

FIG. 5 illustrates another aspect of the system and method of the present invention (10) for monitoring the condition of livestock (12), particularly, for determining the location and controlling the movement of livestock (12). As shown in FIG. 3, the present invention (10) for determining the location and controlling the movement of livestock (12) comprises six primary components, including but not limited to: (1) at least one UAV (14) and/or UAS; (2) an animal locator and herding device(s) (48) onboard the UAV and/or located remotely from the UAV; (3) a transmitter (18) onboard the UAV; (4) a receiver (22) for receiving animal location data from the transmitter; (5) a server (20) for receiving the animal location data from the receiver and further connected to a computer system; and (6) a display (24) for viewing in real-time animal location data obtained from the animal locator and herding device(s) for determining the location and controlling the movement of livestock on a farm or ranch.

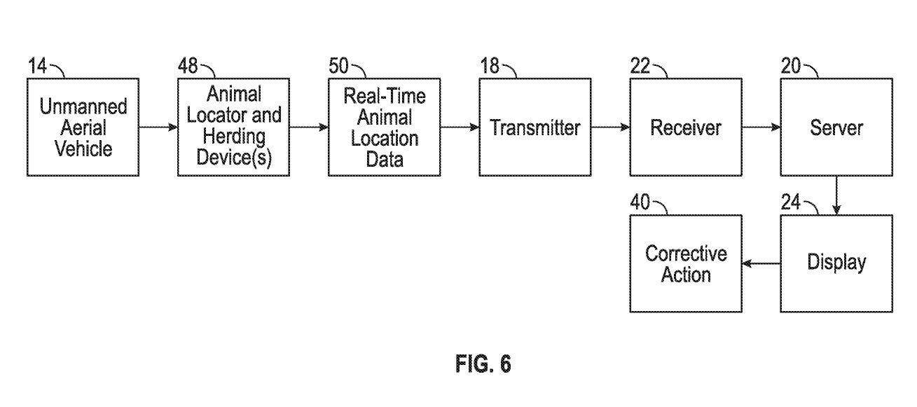

Illustrated in FIGS. 5-6, the first primary component of the present invention (10) for determining the location and controlling the movement of livestock comprises at least one UAV (14) or UAS. The UAV (14) may be of a type standardly used in the industry. Depending upon the intended use of the livestock monitoring system (10) (i.e., whether for use on a large farm/ranch or a confined feedlot), a specific type of UAV (14) may be chosen by an operator (28) (e.g., farm or ranch manager). As mentioned previously, if the intended use is for a smaller area the operator may choose a rotary UAV that typically has between two to ten rotors. Rotary UAVs have limited battery efficiency and are therefore best utilized for relatively smaller areas (e.g., less than 100 acres). These rotors provide optimal stability, control and maneuverability for individual animal assessment on a feedlot, confinement building, pasture, or smaller area. Alternatively, if the intended use is for a large area covering many acres the operator may choose a fixed-wing and/or a blended fuselage-wing UAV such as an all-lifting body. A fixed-wing UAV operates like a small model airplane and may be fabricated using lightweight foam. Because of its minimal weight, a fixed-wing UAV is more efficient in battery usage and is therefore best utilized for larger areas (e.g., over 100 acres) and may travel at speeds in excess of 100 mph. Both rotary and fixed-wing UAVs, used alone or in combination, may be incorporated into the system and method of the present invention for monitoring the health and welfare of livestock. It is to be understood that the precise type and style of UAV is not a limitation to the present invention. The foregoing UAVs are described for illustrative purposes only as it is contemplated other UAVs commonly used in the industry may also be used by the system and method of the present invention.

As shown in FIGS. 5-6, the second primary component of the system and method of the present invention (10) for determining the location and controlling the movement of livestock (12) comprises the animal locator and herding device(s) (48). The animal locator and herding device device(s) (48) may be onboard the UAV (12), wherein the animal locator and herding device(s) (48) may comprise one or more camera(s) (30) for capturing still images and video. The animal locator and herding device(s) may further comprise a plurality of sensors (32) onboard the UAV (14) for determining the location and controlling the movement of livestock (12). The animal locator and herding device(s) (48) may also include remote sensors (34), wherein remote sensors (34) may be located in confinement buildings, corrals, feeding outlets, watering outlets, pastures, and/or combinations thereof. The remote sensors (34) may comprise unique identifiers associated with a particular location and/or purpose for the remote sensor. The remote sensors (34) may also be connected via a bus architecture so that additional sensors may be added or removed as required. The remote sensors (34) may be reusable so that they can be reprogrammed and used at another location or for another purpose. It is contemplated that an array of cameras (30) and sensors (32, 34) in a variety of locations may be utilized as animal locator and herding device(s) (48) by the present invention, including but not limited to, alarms and sirens for startling and herding livestock (12), electric prods for moving livestock (12), electro-optical/infrared imaging, thermal imaging, high definition video and still imaging, multiple object tracking, geo-location, hyperspectral imaging, RFID tags (e.g., ear tags, implants), high frequency tags (e.g., ear tags, implants), spatio-temporal image change detection, GPS, and target tracking.

The animal locator and herding device(s) (48) may obtain real-time animal location data (50) for any particular animal of a livestock herd in a feed lot, confinement building or pasture daily, hourly and/or multiple times per day/night Animal location data (50) may include, but is not limited to, still images and video captured by the one or more camera(s) and information obtained from the plurality of sensors (32) and remote sensors (34). For instance, the operator (28) may be able to identify animals in distress, locate stray animals, and identify specific animals for further observation (52). Furthermore the animal locator and herding device(s) (48) in combination the at least one UAV (14) may be used to herd livestock (12). For example, the operator (28) may control a plurality of UAVs (14) with animal locator and herding device(s) (48) comprising sirens, alarms, and electric prods to create controlled movement (54) of the livestock herd and/or individual animals between pens, between confinement buildings, between pastures, and for loading, shipping and transportation purposes.

As further shown in FIGS. 5-6, the third primary component of the system and method of the present invention (10) for determining the location and controlling the movement of livestock (12) comprises the transmitter (18) (or transceiver). The transmitter (18) may be onboard the UAV (14) and wirelessly communicate the animal location data (50) obtained from the animal locator and herding device(s) (48). As mentioned previously, wireless transmitters utilized in the present invention may be any commercially available type, wherein the precise wireless transmitter not being a limitation of the present invention. The transmitter (18) may include a built-in antennae for transmission of the animal location data (50) obtained from the animal locator and herding device(s) (48). The UAV (14) may further comprise a processor and a guidance system (not shown). The processor may comprise means for performing object detection and/or tracking, and further comprise means for on-board processing of the animal location data (50) prior to transmission.

As further shown in FIGS. 5-6, the fourth primary component of the system and method of the present invention (10) for determining the location and controlling the movement of livestock (12) comprises the receiver (22) (or transceiver). The receiver (22) may wirelessly receive the animal location data (50) communicated from the transmitter (18) onboard the UAV (14) via a local wireless link and/or using a satellite link. The remote sensors (34) may also be wirelessly linked to the receiver (22). If the receiver (22) is a transceiver, the transceiver may wirelessly send commands from the operator (28) via the computer system (38) for operating the guidance system of the UAV (14) and animal locator and herding device(s) (48), wherein the processor onboard the UAV (14) may execute the received commands.

As further shown in FIGS. 5-6, the fifth primary component of the system and method of the present invention (10) for determining the location and controlling the movement of livestock (12) comprises the server (20). The server (20) may be connected wirelessly or via cables to the receiver (22). The receiver (22) may communicate the health and welfare data (36) received from the transmitter (18) to the server (20). The server (20) may be connected to the computer system (38), wherein the operator (28) may transmit commands via the computer system (38) to the guidance system of the UAV (14) for maneuvering the UAV (e.g., adjusting altitude, speed, heading, and positioning) and controlling the animal locator and herding device(s) (48). UAVs (14) of the present invention may be controlled by the operator (28) at all times or have built-in control and/or guidance systems to perform low level human pilot duties such as speed and flight path stabilization, and simple automated navigation functions such as waypoint following.

As further shown in FIGS. 5-6, the sixth primary component of the system and method of the present invention (10) for determining the location and controlling the movement of livestock (12) comprises a display (24) for viewing in real-time the animal location data (50) obtained by the animal locator and herding device(s) (48). The display (24) may be connected to the computer system (38), wherein the computer system (38) may be configured to automatically analyze and selectively create a concise summary and visualization on the display (24) that highlights notable events concerning the livestock herd in a feed lot, confinement building or pasture. The computer system (38) may further comprise a memory (not shown) for storing the animal location data (50) obtained from the animal locator and herding device(s) (48). Examples of computer systems (38) that may be utilized by the livestock monitoring system and method of the present invention (10) include, but are not limited to, a mainframe, a personal computer (PC), a cable set-top box, a television microprocessor, a handheld computer, a lap-top computer, a tablet, a smart-phone device, and/or combinations thereof. The server (20) and computer system (38) may be connected to a satellite or a network such as the Internet or a local area network.

After viewing on the display (24) the animal location data (50) obtained by the animal locator and herding device(s) (48), the operator (28) may take corrective action to protect and/or move livestock (12) on a farm or ranch.

All aspects of the livestock monitoring system and method of the present invention (10) may be used alone or in combination. The livestock monitoring system of the present invention and method of monitoring livestock (10) are universally applicable to farms and ranches of all shapes, sizes, and locations. Thus, the livestock monitoring system and method of the present invention (10) allows the operator (28) to monitor the condition of livestock (12), monitor the condition of feed and water (44), locate animals (52) and move livestock (54) from the convenience of a farm/ranch office (56) without requiring the operator (28) to physically inspect livestock (12) or rely upon additional personnel. Furthermore, while intended for beef cattle, the livestock monitoring system and method of monitoring livestock (12) of the present invention (10) may be used for all manner of livestock (12), including dairy cattle, sheep, swine, goats, poultry, horses and all manner of domesticated or undomesticated livestock. Although the invention has been described and illustrated with respect to preferred aspects thereof, it is not to be so limited since changes and modifications may be made therein which are within the full intended scope of the invention.

What is claimed is:

1. A system for monitoring the condition of livestock, comprising:
at least one unmanned aerial vehicle;
a health and welfare assessment device onboard the unmanned aerial vehicle;
real-time health and welfare data obtained from the health and welfare assessment device onboard the unmanned aerial vehicle;
a transmitter onboard the unmanned aerial vehicle;
a server for receiving the real-time health and welfare data from the transmitter, wherein the server further receives operating instructions for the UAV and the health and welfare assessment device;
a display for viewing in real-time the health and welfare data obtained from the health and welfare assessment device; and
corrective action to safeguard the health and welfare of livestock in response to viewing on the display the real-time health and welfare data obtained by the health and welfare assessment device.

2. The system for monitoring the condition of livestock of claim 1, wherein the health and welfare assessment device onboard the unmanned aerial vehicle comprises electro-optical/infrared imaging, thermal imaging, high definition video and still imaging, multiple object tracking, geo-location, atmospheric soundings, soil moisture determination, biological phenomena observation, barometric pressure recordings, temperature recordings, humidity recordings, meteorological recordings, chemical determination, laser spectroscopy, hyperspectral imaging, RFID tags, high frequency tags, gas analyzers, spatio-temporal image change detection, precision agriculture, pest detection, GPS, target tracking, pH determination, pollution monitoring, plant identification, or combinations thereof.

3. The system for monitoring the condition of livestock of claim 2, wherein the real-time health and welfare data obtained from the health and welfare assessment device onboard the unmanned aerial vehicle comprises:
a) assessing the body temperature of livestock;
b) assessing the onset of disease in livestock;
c) determining the identity of disease in livestock;
d) assessing the contiguousness of disease in livestock;
e) assessing treatment results of diseased livestock; and
f) quarantine monitoring of diseased livestock.

4. The system for monitoring the condition of livestock of claim 2, wherein the real-time health and welfare data obtained from the health and welfare assessment device onboard the unmanned aerial vehicle comprises:
a) assessing bedding availability and cleanliness for livestock;
b) assessing mineral offerings for livestock;
c) determining drug requirements for livestock;
d) detecting fertility status in livestock;
e) assessing the pH of biological fluids from livestock;
f) assessing blood flow or blood oxygenation of livestock;
g) assessing vocalization and respiration recognition of livestock;
h) assessing breath and saliva contents from livestock;
i) identifying excessive livestock behaviors;
j) identifying livestock downers; or
k) combinations thereof.

5. The system for monitoring the condition of livestock of claim 2, wherein the real-time health and welfare data obtained from the health and welfare assessment device onboard the unmanned aerial vehicle comprises:
a) weather conditions;
b) environmental temperatures; and
c) biosecurity surveillance.

6. The system for monitoring the condition of livestock of claim 2, wherein the real-time health and welfare data obtained from the health and welfare assessment device onboard the unmanned aerial vehicle comprises:
a) calculating the rate of gain of livestock;
b) identifying eating patterns of livestock;
c) identifying water intake levels of livestock; and
d) identifying eating disorders in livestock.

7. The system for monitoring the condition of livestock of claim 1, further comprising:
a feed and water assessment device onboard the unmanned aerial vehicle for monitoring feed and water conditions in a feed lot, confinement building or pasture;
real-time feed and water data obtained from the feed and water assessment device onboard the unmanned aerial vehicle; and
corrective action to promote the growth and vitality of livestock in response to viewing on the display the real-time feed and water data obtained by the feed and water assessment device;
wherein the server receives the real-time feed and water data from the transmitter onboard the unmanned aerial vehicle, wherein the server further receives operating instructions for the feed and water assessment device.

8. The system for monitoring the condition of livestock of claim 7, wherein the feed and water assessment device onboard the unmanned aerial vehicle comprises electro-optical/infrared imaging, thermal imaging, high definition video and still imaging, multiple object tracking, geo-location, temperature recordings, humidity recordings, chemical determination, laser spectroscopy, hyperspectral imaging, RFID tags, high frequency tags, gas analyzers, spatio-temporal image change detection, precision agriculture, pest detection, GPS, target tracking, pH determination, pollution monitoring, plant identification, or combinations thereof.

9. The system for monitoring the condition of livestock of claim 8, wherein the real-time feed and water data obtained from the feed and water assessment device onboard the unmanned aerial vehicle comprises:
a) monitoring the proper distribution of feed to livestock;
b) monitoring feed delivery patterns to livestock;
c) identifying the amount of feed available to livestock, at any given time, at any given location, and at any specific time of day/night;
d) observing livestock response in relationship to feed delivery;
e) determining feed availability to livestock;
f) determining feed and water cleanliness available to livestock;
g) determining feed and water quality available to livestock;
h) determining the freshness of feed available to livestock; and
i) determining water cleanliness available to livestock.

10. The system for monitoring the condition of livestock of claim 7, further comprising:
an animal locator and herding device onboard the unmanned aerial vehicle for determining the location and controlling the movement of livestock;
real-time animal location data obtained from the animal locator and herding device onboard the unmanned aerial vehicle; and
corrective action to protect and/or move livestock in response to viewing on the display the real-time animal location data obtained by the animal locator and herding device;
wherein the server receives the real-time animal location data from the transmitter onboard the unmanned aerial vehicle, wherein the server further receives operating instructions for the animal locator and herding device.

11. The system for monitoring the condition of livestock of claim 10, wherein the animal locator and herding device onboard the unmanned aerial vehicle comprises alarms and sirens for startling and herding livestock, electric prods for moving livestock, electro-optical/infrared imaging, thermal imaging, high definition video and still imaging, multiple object tracking, geo-location, hyperspectral imaging, RFID tags, high frequency tags, spatio-temporal image change detection, GPS, target tracking, or combinations thereof.

12. The system for monitoring the condition of livestock of claim 11, wherein the real-time animal location data obtained from the animal locator and herding device onboard the unmanned aerial vehicle comprises:
a) identifying livestock in distress;
b) locating stray livestock; and
c) identifying specific animals for further observation.

13. The system for monitoring the condition of livestock of claim 11, wherein the real-time animal location data obtained from the animal locator and herding device onboard the unmanned aerial vehicle comprises:
a) controlling a plurality of UAVs with animal locator and herding device(s); and
b) creating controlled movement of the livestock herd and/or individual animals;
c) wherein the plurality of UAVs comprise sirens, alarms, and/or electric prods.

14. The system for monitoring the condition of livestock of claim 10, further comprising a health and welfare assessment device, a feed and water assessment device, and/or an animal locator and herding device remote from the unmanned aerial vehicle.

15. A system for monitoring the condition of livestock, comprising:
at least one unmanned aerial vehicle;
a health and welfare assessment device, a feed and water assessment device, and an animal locator and herding device onboard the unmanned aerial vehicle;
real-time health and welfare data obtained from the health and welfare assessment device onboard the unmanned aerial vehicle;
real-time feed and water data obtained from the feed and water assessment device onboard the unmanned aerial vehicle;
real-time animal location data obtained from the animal locator and herding device onboard the unmanned aerial vehicle;
a transmitter onboard the unmanned aerial vehicle;
a server for receiving the real-time health and welfare data, real-time feed and water data, and real-time animal location data from the transmitter, wherein the server further receives operating instructions for the UAV and the health and welfare assessment device, the feed and water assessment device, and the animal locator and herding device;
a display for viewing in real-time the health and welfare data, the feed and water data, and the animal location data; and
corrective action to safeguard the health and welfare of livestock in response to viewing on the display the real-time health and welfare data obtained by the health and welfare assessment device;
corrective action to promote the growth and vitality of livestock in response to viewing on the display the real-time feed and water data obtained by the feed and water assessment device; and
corrective action to protect and/or move livestock in response to viewing on the display the real-time animal location data obtained by the animal locator and herding device.

16. The system for monitoring the condition of livestock of claim 15, wherein the real-time health and welfare data obtained from the health and welfare assessment device onboard the unmanned aerial vehicle comprises:
a) assessing the body temperature of livestock;
b) assessing the onset of disease in livestock;
c) determining the identity of disease in livestock;
d) assessing the contiguousness of disease in livestock; and
e) quarantine monitoring of diseased livestock;
wherein the real-time feed and water data obtained from the feed and water assessment device onboard the unmanned aerial vehicle comprises:
f) monitoring the proper distribution of feed to livestock;
g) monitoring feed delivery patterns to livestock;
h) identifying the amount of feed available to livestock, at any given time, at any given location, and at any specific time of day/night;
i) observing livestock response in relationship to feed delivery;
j) determining feed availability to livestock;
k) determining feed and water cleanliness available to livestock;
l) determining feed and water quality available to livestock;
m) determining the freshness of feed available to livestock; and
wherein the real-time animal location data obtained from the animal locator and herding device onboard the unmanned aerial vehicle comprises:
n) identifying livestock in distress;
o) locating stray livestock; and
p) identifying specific animals for further observation.

17. The system for monitoring the condition of livestock of claim 16, wherein the health and welfare assessment device onboard the unmanned aerial vehicle comprises:
electro-optical/infrared imaging, thermal imaging, high definition video and still imaging, multiple object tracking, temperature recordings, humidity recordings, and combinations thereof;
wherein the feed and water assessment device onboard the unmanned aerial vehicle comprises: high definition video and still imaging, chemical determination, laser spectroscopy, hyperspectral imaging, pest detection, pH determination, pollution monitoring, plant identification, and combinations thereof; and
wherein the animal locator and herding device onboard the unmanned aerial vehicle comprises: alarms and sirens for startling and herding livestock, geo-location, high definition video and still imaging, multiple object tracking, GPS, and combinations thereof.

18. A method for monitoring the condition of livestock, comprising:
providing at least one unmanned aerial vehicle;
providing a health and welfare assessment device onboard the unmanned aerial vehicle;
obtaining real-time health and welfare data from the health and welfare assessment device onboard the unmanned aerial vehicle;
transmitting the real-time health and welfare data to a server using a transmitter onboard the unmanned aerial vehicle;
receiving on the server the real-time health and welfare data sent from the transmitter;
viewing in real-time on a display the health and welfare data obtained from the health and welfare assessment device; and
taking corrective action to safeguard the health and welfare of livestock in response to viewing on the display the real-time health and welfare data obtained by the health and welfare assessment device.

19. The method of claim 18, further comprising:
providing a feed and water assessment device onboard the unmanned aerial vehicle for monitoring feed and water conditions in a feed lot, confinement building or pasture;
obtaining real-time feed and water data obtained from the feed and water assessment device onboard the unmanned aerial vehicle;
transmitting the real-time feed and water data to a server using a transmitter onboard the unmanned aerial vehicle;
receiving on the server the real-time feed and water data sent from the transmitter;
viewing in real-time on a display the feed and water data obtained by the feed and water assessment device; and
taking corrective action to promote the growth and vitality of livestock in a feed lot, confinement building or pasture.

20. The method of claim 19, further comprising:
providing an animal locator and herding device onboard the unmanned aerial vehicle for determining the location and controlling the movement of livestock;
obtaining real-time animal location data obtained from the animal locator and herding device onboard the unmanned aerial vehicle; and
transmitting the real-time animal location data to a server using a transmitter onboard the unmanned aerial vehicle;
receiving on the server the real-time animal location data sent from the transmitter;
viewing in real-time on a display the animal location data obtained by the animal locator and herding device; and
taking corrective action to protect and/or move livestock in response to viewing on the display the real-time animal location data obtained by the animal locator and herding device.
